(19) United States
(12) Patent Application Publication
(10) Pub. No.: US 2006/0178674 A1
(43) Pub. Date: Aug. 10, 2006

(54) Surgical apparatus having configurable portions

(76) Inventor: John McIntyre, Rancho Santa Margarita, CA (US)

Correspondence Address: STETINA BRUNDA GARRED & BRUCKER, 75 ENTERPRISE, SUITE 250, ALISO VIEJO, CA, 92656, US

(21) Appl. No.: 11/346,765

(22) Filed: Feb. 3, 2006

Related U.S. Application Data
(60) Provisional application No. 60/650,911, filed on Feb. 8, 2005.

(51) Int. Cl. A61F 11/00

(52) U.S. Cl. 606/108

(57) ABSTRACT

A device for performing a surgical procedure within a body and a method for utilizing such a device to treat a patient's heart with minimal invasiveness. The device comprises a handle, a guide shaft, and a sleeve in slideable connection with the guide shaft. The guide shaft is connected to the handle at a proximal portion and has a first curved position at the distal portion. Slideable manipulation of the sleeve changes the first curved position. The method comprises providing such a device, introducing the device into a minimally invasive port in the patient, and providing treatment to the patient's heart.

### CROSS-REFERENCE TO RELATED APPLICATIONS

This utility patent application claims priority to Provisional Patent Application Ser. No. 60/650,911 entitled SURGICAL APPARATUS HAVING CONFIGURABLE PORTIONS which was filed on Feb. 8, 2005.

### STATEMENT RE: FEDERALLY SPONSORED RESEARCH/DEVELOPMENT

Not Applicable

### BACKGROUND

The present invention relates to the field of surgery, and, more particularly to surgical procedures and apparatus for use with minimally invasive ports.

Medical science has developed a wide variety of surgical devices for assessing different areas of the body and for performing various surgical procedures. Many of these surgical devices and procedures require a relatively large opening within the patient's body just to gain access into the body cavity and to the desired area of treatment. This is particularly true with surgical devices having broad shapes and configurations and when used in conjunction with procedures requiring large movements of the device or requiring multiple treatment locations. Improvements are continuously being sought to reduce the negative effects of any surgery, including the effects incurred when penetrating the body with surgical devices and further when accessing the treatment areas. This concern has lead to the desire to reduce such effect and further to utilize minimally invasive procedures wherever possible.

In minimally invasive surgical procedures, the trauma to the body is reduced, in part, by reducing the surgical opening required into the body cavity. Thus, there is a great desire to modify existing open surgical devices for application with minimally invasive procedures and ports.

Transmyocardial revascularization (“TMR”) is a procedure wherein energy is delivered to a region of the heart in order to create channels across the wall of the heart. The procedure is typically performed in patients suffering from severe angina. TMR is performed in a surgical setting in which access to the left ventricle is typically gained through an open surgical sternotomy or a thoracotomy. With the advent of minimally invasive thoracoscopic surgical procedures in recent years, it is desirable to deliver TMR therapy minimally invasively via ports. Present TMR surgical devices, however, require unique configurations that generally preclude the use of such minimally invasive ports.

### BRIEF SUMMARY

The present invention comprises a surgical device for treating living tissue within a body. The device includes a handle portion for gripping and manipulation by the surgeon or other user. An elongated tubular guide shaft is connected to the distal handle portion and extends outwardly into a distal portion having a curved shape. The distal end of the guide shaft includes a head assembly that is adapted for forming a contact surface with a desired region of the heart and includes a treatment assembly that is adapted for the desired treatment. The device includes an advancement mechanism to move the treatment assembly relative to the head assembly. The advancement mechanism allows the treatment assembly to be translated along the distal portion of the guide shaft so as to translate outwardly from the head assembly and also to be retracted.

A tubular guide shaft straightening assembly is slideably connected to the guide shaft wherein translation of the straightening assembly along the guide shaft elongates the curved distal portion of the guide shaft and straightens out the curved shape of the guide shaft. Retracting the straightening assembly allows the guide shaft to return to a curved shape.

The present invention further comprises a minimally invasive procedure for treating a patient's heart. The method comprises the steps of providing an elongated surgical device having a guide shaft extending away from a handle portion into a curved distal portion and including a treatment assembly. A sleeve assembly is slideably mounted over the guide shaft and configured such that when the sleeve is extended along the guide shaft and away from the handle the curved distal portion of the guide shaft is elongated along the axis of the guide shaft and the curvature reduced or eliminated such that the guide shaft is aligned along a single axis. The curvature of the guide shaft is reformed when the sleeve is retracted towards the handle.

In the procedure, the sleeve is first slid outwardly along the guide shaft from a normally retracted position so as to reduce the curvature of the distal portion and straighten out the entire length of the guide shaft. The guide shaft is then introduced into a minimally invasive port in the patient. Once the elongated guide shaft is positioned within the patient, the sleeve may be retracted relative to the guide shaft, returning the distal portion into a curved configuration for treating the heart. This step, including extending and retracting the sleeve, may be repeated during the procedure so as to reconfigure the guide shaft whenever desired. The treatment assembly is then manipulated into position adjacent the heart such that the desired region of the heart or associated tissue may be treated.

The present invention further comprises a system and method of surgical myocardial revascularization of the myocardium of the heart of a patient. In this procedure, a surgical opening and preferably a minimally invasive port is created within the patient. An elongated flexible surgical apparatus configured into an insertion configuration is inserted into the surgical opening and directed into the chest cavity. The surgical apparatus preferably includes a lasing mechanism in connection with a surgical lasing tip located at the distal end of a flexible guide shaft. The guide shaft, including the lasing tip is then guided within the patient and into a desired area within the chest cavity. The elongated guide shaft portion of the surgical apparatus may be adjusted and configured between a straightened shaft configuration and a configuration having a curved distal portion so as to facilitate advancement and positioning within the patient. By manipulating a sleeve on the apparatus, the guide shaft can be reconfigured from an essentially straight configuration into a curved configuration having an essentially ninety degree curvature or anywhere in between. In addition, the guide shaft, including the attached surgical end may be rotated to further assist in the advancement and positioning of the surgical end adjacent the regions of the heart to be treated. The heart is next irradiated with laser energy emitted from the lasing apparatus with sufficient energy and for a sufficient time to cause a channel to be formed from the exterior surface of the epicardium through the myocardium and the epicardium.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a side view of an embodiment of the surgical device of the present invention.

FIG. 2A is a side view of a preferred embodiment of the surgical device of the present invention with the straightening sleeve removed.

FIG. 2B is a top view of an embodiment of the surgical device of the present invention with the sleeve removed and having an enlarged cup.

FIG. 2C is a side view of an embodiment of the surgical device of the present invention with the sleeve removed and having an enlarged cup.

FIG. 2D is a bottom view of an embodiment of the surgical device of the present invention with the sleeve removed and having an enlarged cup.

FIG. 3 is a side view of a preferred embodiment of the surgical device of the present invention showing the straightening sleeve in a retracted position.

FIG. 4 is a side view of a preferred embodiment of the surgical device of the present invention showing the straightening sleeve in the extended position.

### DETAILED DESCRIPTION

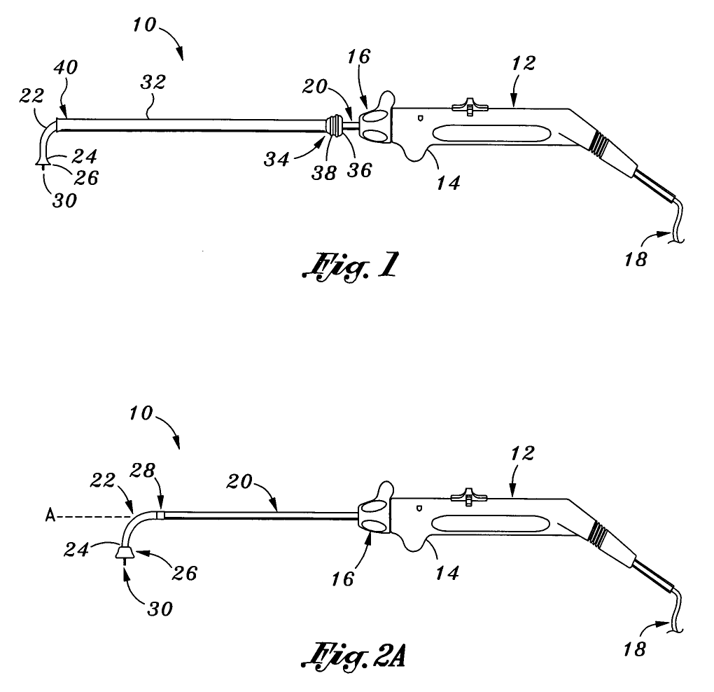

While a variety of embodiments of the present invention are disclosed herein, one exemplary and the presently preferred embodiment of the surgical device is illustrated generally as reference number 10 in FIGS. 1-4. This embodiment of the surgical device 10 is particularly suitable for procedures for treating the heart, including transmyocardial revascularization, biopsy and related procedures. The device 10 is adapted for hand use and manipulation and may be held in several positions or with both hands.

A preferred method using the device 10 of the present embodiment involves using a treatment assembly associated with the device to perforate the epicardium of the heart to create myocardial revascularization pathways. Such pathways are typically revascularization channels which extend into myocardium and may or may not communicate with the ventricle.

Referring now to FIG. 1, the preferred mechanical surgical device 10 includes a hand piece 12 which is a housing molded or machined from a plastic material, and defining a contoured surface 14 defining one or more finger grip indentations. Preferably, the contoured surface 14 provides tactile feedback regarding the position of the hand on the device so the physician need not look away from the medical procedure or other task at hand. The contoured surface 14 further assists the user to securely hold the hand piece without slippage in at least two, different positions during either left or right handed operation of the device 10. A neck portion or nosecone 16 extends from the hand piece 12 and is preferably a separate component to allow for a rotary connection with the hand piece. The nosecone 16 may also be constructed from a molded or machined plastic or similar to the hand piece 12, may be constructed from other materials such as metal or composite materials. The hand piece 12 and nosecone 16 include a continuous passageway for supporting a treatment assembly 18 which in a preferred TMR procedure includes an optical fiber bundle whose proximal end is connected to a laser energy source (not shown). In the preferred embodiment, the treatment assembly is moveable within the apparatus passageway using a mechanical finger slide configured as part of the handle portion 12 but may also be moveable using most any form of mechanical, electro mechanical slide mechanism or may even be moved using an automated mechanism.

A tubular guide shaft 20 extends outwardly from a proximal portion connected to the nosecone 16 of the handle 12 into a distal curved portion 22. The proximal portion of the guide shaft 20 may be constructed of metal, plastic or composite materials and may be slightly malleable to allow some flexibility. Preferably, the proximal portion of the guide shaft is made from a medical grade tubular stainless steel and the distal curved portion from a flexible plastic with memory. The guide shaft 20 includes a lumen having an opening or diameter sufficient to allow passage of the desired treatment apparatus 18. The guide shaft 20 is rigidly attached to the nosecone 16 and may be rotated about the axis “A” by twisting or otherwise rotating the nosecone relative to the handle portion 12.

As shown, the guide shaft 20 extends away from the handle 12 to the distal portion 22 having a curved shape of approximately 90 degrees from the axis “A”. The curved distal portion 22 of the guide shaft 20 terminates at distal end 24 which is connected to a protective and stabilizing tip assembly 26. The stabilizing tip 26 is generally ball, cup or disc shaped and is designed to contact tissue and maintain contact of the device 10 on the region of tissue being treated. The stabilizing tip 26 may be constructed from generally yieldable materials such as silicone, soft elastic, rubber or foam and may also be metallic or plastic. The stabilizing tip 26 may be part of or permanently attached to the shaft 20 or may be detachable with conventional snap-mount or screw mount mechanisms. Different detachable stabilizing tips 26, such as suction and drug delivery tips, may be provided to accommodate different treatment procedures as well as differing access ports. The distal end or tissue contacting surface of the tip 26 may be textured to provide a gripping surface, and suction may be provided at the proximal end of the hand piece to extend through the shaft 20 to further secure the stabilizing tip 26 to the tissue being treated, including the heart. The stabilizing tip 26 includes a bore aligned and in connection with the bore formed through the guide shaft 20. In this way, the treatment assembly 18 may freely pass through the guide shaft 20 and the cup tip 26.

In this preferred embodiment configured for TMR, the stabilizing tip 26 is a flexible cup having a proximal end with an outer diameter equivalent to the outer diameter of the distal end 24 of the guide shaft 20. The proximal end of the cup 26 is rigidly bonded to the distal end 24 of the guide shaft 20 and may be configured with a proximal end diameter similar to that of the distal end 24 of the guide shaft 20. The cup 26 tapers outwardly from the guide shaft 20 into a contact surface outer diameter of about 8 mm. The cup 26 is preferably made from a medical grade polyether block co-polyamide polymer (“Pebax”) or other medical grade nylon type material that is sufficiently pliable to form a contact surface with the tissue being treated and is bonded to the distal end 24 of the guide shaft 20. Alternatively, the cup 26 and the curved portion 22 of the guide shaft 20 may be formed from a single piece of flexible plastic material such as Pebax. Alternatively, the guide shaft 20 and the cup 26 may be constructed from multiple segments of material having varying hardness. For example, a stiffer material may be used to create a stiffer, or more spring like curved portion 22 and a softer material may be used to create an atraumatic cup 26.

The present invention advantageously decreases the outer diameter of the tissue contacting surface of the cup 26 as compared to present configurations of TMR surgical devices. The smaller outer diameter facilitates access to difficult-to-reach areas of the heart when moving to a new location to create a channel. In addition, the smaller diameter facilitates use of the device 10 with smaller and minimally invasive ports. As designed, the smaller diameter cup still allows for sufficient stabilization and further ensures that the distal end of the treatment assembly contacts the treatment area at the desired angle. In the apparatus shown, the cup 26 is designed for proper stabilization against the epicardium and further ensures that the lasing treatment tip 30 contacts and enters the epicardium at an approximate perpendicular angle to the tissue surface.

The curved portion 22 is configured to have and naturally retain a curvature of between 30 and 150 degrees and preferably about 90 degrees from the elongated axis of the guide shaft 20. When the device 10 is provided with a rotatable neck portion 16, the orientation of the curved portion 22 and the cup 26 may be altered by rotating the neck portion relative to the handle 12. Rotation mechanisms between the neck portion 16 and the handle portion 12 may include conventional spring fingers, detents and ratchet assemblies or simply a friction fit. In addition, the neck portion 16 is provided with a configuration that facilitates gripping while rotating.

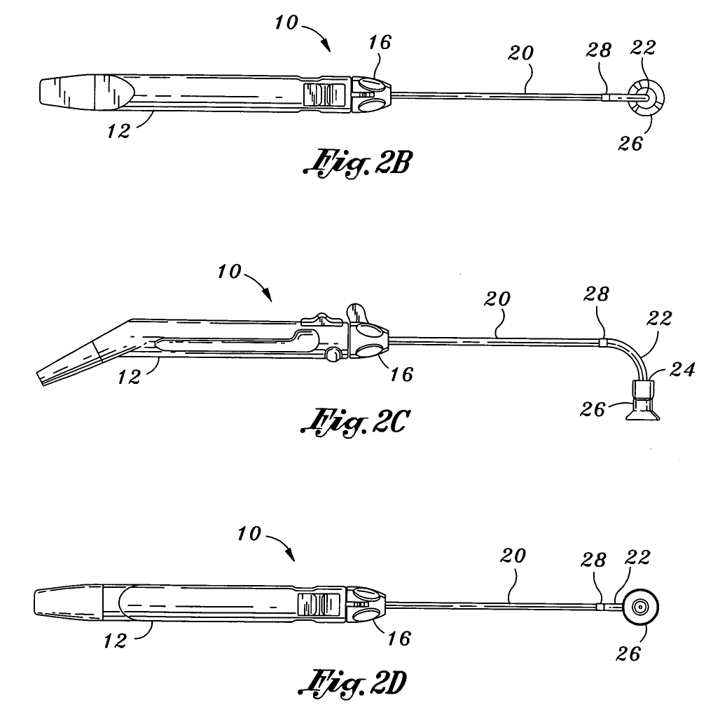

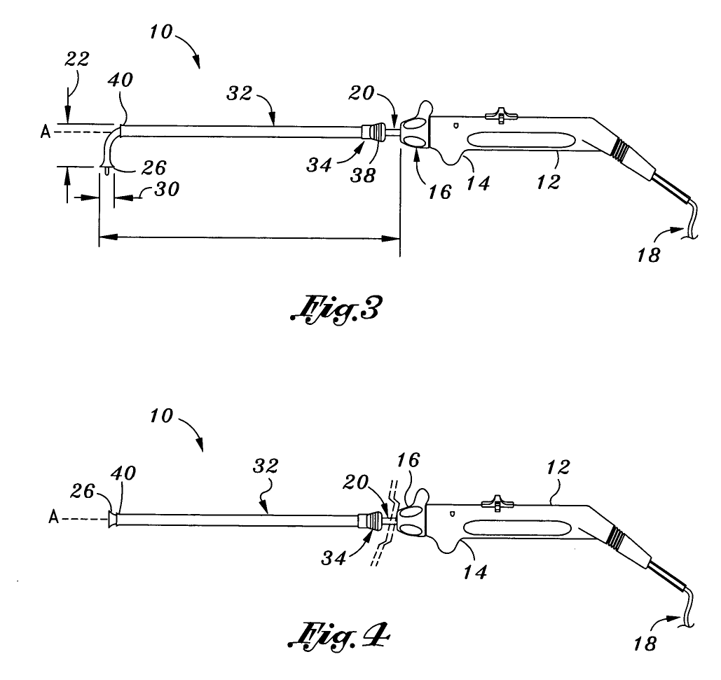

Referring now to FIGS. 2A-2D, the curved portion 22 of the guide shaft, in the preferred embodiment is made from a flexible plastic material such as a tubular section of Pebax. The Pebax curved portion 22 is bonded at a proximal end 28 to the generally straight proximal section of the guide shaft 20 and also bonded at its distal end 24 to the cup 26. Other methods of securing the multiple sections of the guide shaft 20 may also be used. Alternatively, the entire guide shaft may be made from a singular piece of flexible material such as Pebax, other nylon, silicone, or most any other surgical grade material or combination of such materials. In addition, the guide shaft may be made from multiple segments, each having different qualities such as hardness, colorings and markings. For example, the guide shaft 20 may be made from materials having differing flexibility, elasticity as well as memory. This way, one can adjust the amount of force required to straighten out the curved portion 22 or even the amount of spring force the curved portion exerts to return to its curved shape when it is straightened out. Similarly, various colorings may be provided as positional locators along the length of the guide shaft. These colorings may be provided as differing materials or simply as external markings.

In the preferred configuration described, the guide shaft 20 is about 22 centimeters long, including the proximal portion (straight section) made from a 304 stainless steel tubing of about 18.5 centimeters in length bonded to the curved section of Pebax tubing having an approximate length of 3.3 centimeters. The passageway extending through the surgical device 10 is adapted to allow for the passage of a treatment assembly 18.

In general, the curved portion 22 of the guide shaft 20 is specifically designed and adapted to be flexible between the curved shape 22 having an angle of about 90 degrees from axis “A” and an elongated straightened configuration wherein the curved portion is straight and generally aligned along axis “A”. Likewise, the portion of the treatment assembly 18 passing through the guide shaft is also configured to be flexible such that it can also transition between an essential 90 degree curve and a straight path.

Referring now back to FIGS. 1 and 3, a straightening sleeve 32 is mounted about and coaxial with the guide shaft 20. In the embodiment shown, the straightening sleeve 32 is a tube including a proximal portion 34 configured as a gripping section that is mounted over the shaft 20 adjacent the handle 12. The gripping section 34 includes a bearing surface 36 along its inner diameter that contacts the guide shaft 20 to facilitate sliding of the sleeve 32 along the guide shaft 20. Preferably, the bearing surface 36 also reduces lateral movement of the sleeve 32 relative to the guide shaft 20. The gripping section 34 may also include a configuration that facilitates friction against the user's fingers while sliding the sleeve relative to the guide shaft 20. In the embodiment shown, the gripping section 34 is formed of a plastic and has a larger outside diameter than the remainder of the sleeve 32. In addition, the gripping section 34 includes several raised rings 38 aligned generally perpendicular to sleeve 32. Alternatively, the sleeve 32 and the gripping section 34 may be constructed of almost any rigid material and may further include any configuration that facilitates a user's ability to slide the sleeve 32 relative to the guide shaft 20.

In a preferred embodiment, the integral straightening sleeve 32 is composed of a stainless steel tube connected to the tubular gripping section 34 at the proximal end. The sleeve 32 is coaxial with the guide shaft 20. The sleeve 32 is slideable along the guide shaft 20 from a first sleeve position (retracted position) wherein the proximal end of the gripping section 32 is adjacent to the distal end of the neck portion 16 as illustrated in FIGS. 1 and 3. In the fully retracted position, the curved portion 22 is bent at approximately 90 degrees from the elongated axis “A” of the device 10. The straightening sleeve 32 may also be made from various materials or multiple segments of materials and may even be fitted with a special distal end specifically configured for contacting and straightening the curved distal portion 22. In addition, the sleeve 32 may be fitted with various devices to facilitate movement along the shaft 20, such as bushings. The sleeve may also be fitted with a locator device, including a hole for identifying markings or colorings on the guide shaft 20.

Referring now to FIG. 4, the surgical device of the present invention is shown with the straightening sleeve 32 fully extended (extended position) relative to the guide shaft 20 such that the curved portion 22 of the guide shaft is straightened out and the entire guide shaft elongated along the axis “A”. When the sleeve 32 is extended, its distal end 40 is moved along the flexible curved portion 22 of the guide shaft 20 directing it into the sleeve without kinking or damaging it and without distortion of the interior bore defining the passageway through the device 10. The sleeve distal end 40 may be formed into a smooth flanged outer edge to facilitate sliding along the curved portion 22 of the guide shaft and reduce distortion of the cup 26. The sleeve distal end 40 may also be configured to accept the distal end 24 of the guide sleeve or even a treatment end 30.

The straightening sleeve 32 may also be tapered along its elongated axis such that it has a smaller interior diameter at its distal end 40 and retains a tighter fit around the curved portion 22 of the guide shaft 20. A tapered sleeve 32 may reduce distortions to the cup 20 when moved into the extended position. Alternatively, sleeve distal end 40 may be fit with a flange that is specifically made to facilitate the curved portion 22 sliding into the sleeve 32 when extending the sleeve. Moving the sleeve 32 between the retracted position and the extended position, and there between, allows a user to modify the angle of the curved portion 22 from between a curved position of 90 degrees with the axis “A” and a straight guide shaft position.

During a preferred TMR procedure using the surgical device 10 or the present invention, energy is applied to myocardial tissue of the heart by means of the treatment assembly 18 supported within the passageway extending through the hand piece 12 and further extending through the guide shaft 20 and out from the cup 26. In the currently preferred procedure, a laser provides the energy that is directed through the treatment assembly 18 which includes a means of carrying the laser energy to the treatment tip 30. In the preferred embodiment, a fiber optic cable is used.

To facilitate a minimally invasive surgical procedure using the present surgical device 20, including the use of 8 mm ports, the straightening sleeve 32 is moved along the guide shaft 20 towards the curved portion 22 so as to straighten out the curved distal portion. Preferably, the sleeve 32 is slid distally along the guide shaft 20 so that as it is slid over the curved portion 22, the walls of the sleeve force the curved portion to be straightened out inside the sleeve itself. Once the curved portion 22 is straightened out, the guide shaft 20 may be inserted into a minimally invasive port within the patient.

The physician can then manipulate the surgical device 10 and particularly, the guide shaft 20 within the patient. Once the user determines the guide shaft 20 is appropriately positioned, the straightening sleeve 32 may be retracted to increase the curvature of the distal portion 22 of the guide shaft. The user may retract the sleeve 32 sufficient to create the desired curvature 22. Alternatively, the user may retract or extend the sleeve 32 to achieve any desired configuration (curvature) at multiple times during a procedure. In the preferred embodiment of the present invention, the bearing 36 maintains a sufficiently tight friction fit around the proximal portion of the guide shaft 20 so as to maintain its position relative to the guide shaft once it is released by the user.

In a presently preferred TMR procedure, the guide shaft 20 is inserted into the chest cavity of a patient through an 8 mm port. If necessary, the guide shaft 20 may be adjusted from a straight configuration (FIG. 4) into one having a curved configuration (FIG. 3) so as to facilitate passage of the guide shaft within the patient or to facilitate positioning of the guide shaft adjacent the treatment area. Once positioned adjacent the epicardium, the user adjusts the treatment assembly 18 such that the treatment tip 30 is positioned perpendicular to the epicardium. It is often necessary to retract the sleeve 32 such that the curved portion 22 of the guide shaft 20 forms an approximately 90 degree angle (FIG. 3) to create the desired perpendicular positioning of the treatment tip 30 with the epicardium wall. In addition, the nosecone 16 may also be used to rotate the cup 26 and treatment tip 18 as desired.

Further details of the present invention, including various methods of using the present invention may be found with reference to the Detailed Description of Embodiment section of U.S. Pat. No. 5,713,894 issued on Feb. 3, 1998 to Murphy-Chutorian and Harman and to the Detailed Description of the Preferred Embodiment section of U.S. Pat. No. 5,976,164 issued on Nov. 2, 1999 to Bencini et al. of which both are incorporated in their entirety herein by reference.

The foregoing describes the features and benefits of the present inventions in various embodiments. Those of skill in the art will appreciate that the present invention is capable of various other implementations and embodiments that operate in accordance with the foregoing principles and teachings. For example, many of the components may be made from various materials and may be interconnected in various ways. Moreover, the arrangement of an elongated guide shaft having a curved distal portion and a sliding sleeve mechanism for elongating the curved portion into a straight portion may be accomplished by using differing tubular shapes or even with a portion of or all of the straightening sleeve adjacent to the guide shaft rather than slideably mounted over and around it. The curved portion of the guide shaft may also be positioned proximally adjacent the hand piece or even mid section. The curved portion may be curved along three axes. The housing may be made of materials other than plastic and may be configured differently to provide alternative designs. Further, the various features of the embodiments disclosed herein can be used alone, or in varying combinations with each other and are not intended to be limited to the specific combination described herein. Accordingly, this detailed description is not intended to limit the scope of the present invention, which is to be understood by reference the claims below.

What is claimed:

1. A device for performing a surgical procedure within a body, comprising:
a. a handle;
b. a guide shaft having a proximal end in connection with the handle and extending into a distal portion having a first curved position; and
c. a sleeve in slideable connection with the guide shaft wherein slideable manipulation of the sleeve changes the first curved position of the distal portion.

2. The device of claim 1 wherein the guide shaft is tubular.

3. The device of claim 2 wherein the sleeve is tubular and slideable over the guide shaft.

4. The device of claim 3 wherein sliding the sleeve away from the handle straightens out the curved portion of the guide shaft.

5. The device of claim 4 wherein the tubular sleeve is tapered to form an elongated funnel shape.

6. The device of claim 4 wherein retracting the sleeve towards the handle allows the distal portion to return to a first curved position.

7. The device of claim 1 wherein the distal end of the guide shaft includes a tip for placement on a body region subject to the desired surgery.

8. A hand held surgical device for treating heart tissue within a body comprising:
a. a handle portion;
b. a tubular guide shaft having a proximal end connected to the handle portion and extending away from the handle into a curved distal portion;
c. a head portion connected to a distal end of the guide shaft, the head portion forming a contact surface for contacting a desired region of the body;
d. a treatment assembly advanceable along the axis of the distal portion for treating the heart tissue;
e. an advancement mechanism for moving the treatment assembly through the distal end of the guide shaft; and
f. a sleeve portion slideable along the axis of the guide shaft wherein slideable manipulation of the sleeve portion alters the angle of the curved portion of the distal end of the guide shaft.

9. The device of claim 8 wherein the distal portion of the guide shaft is made from a resilient material.

10. The device of claim 8 wherein the distal portion of the guide shaft is made from a polyether block co-polyamide polymer material.

11. The device of claim 8 wherein the curved portion of the guide shaft has a curvature of between 30 and 150 degrees.

12. The device of claim 8 wherein manipulation of the sleeve along the guide shaft completely straightens out the curved portion of the guide shaft.

13. The device of claim 8 wherein the distal portion of the guide shaft is bonded to a proximal portion of the guide shaft.

14. The device of claim 8 wherein retracting the sleeve along the guide shaft does not affect the curved portion of the distal portion and wherein extending the sleeve along the guide shaft straightens out the curved portion of the distal end so as to facilitate entry of the guide shaft through a minimally invasive port.

15. A minimally invasive procedure for treating a patient's heart, comprising the steps of:
a. providing a surgical device having an elongated guide shaft with a flexible curved distal portion in connection with a treatment assembly and a proximal end in connection with a hand portion, the guide shaft having a slideable sleeve wherein sliding the sleeve away from the handle straightens the curved distal portion along the axis of the guide shaft and reduces the curvature and wherein curvature returns when the sleeve is retracted towards the handle;
b. moving the sleeve towards the distal portion of the guide shaft to straighten out the curved distal portion;
c. introducing the distal portion of the elongated surgical device into a minimally invasive port in the patient;
d. positioning the surgical device to a desired position within the patient; and
e. directing the treatment assembly so as to treat the heart.

16. The method of claim 15 further comprising the step of retracting the sleeve to increase the curvature of the distal portion.

17. The method of claim 15 further comprising the step of adjusting the angle of the distal portion of the guide shaft by retracting the sleeve until the desired curvature is achieved.

18. The method of claim 15 further comprising the step of adjusting the angle of the distal portion of the guide shaft by retracting and extending the sleeve until the desired curvature is achieved.
